(19) United States
(12) Patent Application Publication
(10) Pub. No.: US 2007/0128692 A1
(43) Pub. Date: Jun. 7, 2007

(54) Chimeric empty capsids of the infectious bursal disease viruse (ibdv), obtainment process and applications

(76) Inventors: Jose Francisco Rodriguez Aguirre, Madrid (ES); Jose Ruiz Caston, Madrid (ES); Maria Dolores Gonzalez de Llano, Madrid (ES); Maria Dolores Rodriguez Aguirre, Madrid (ES); Soledad Blanco Chapinal, Madrid (ES); Ana Maria Ona Blanco, Madrid (ES); Irene Saugar Gomez, Madrid (ES); Fernando Abaitua Elustondo, Oxted (GB); Daniel Luque Buzo, Madrid (ES); Juan Ramon Rodriguez Fernandez-Alba, Madrid (ES)

Correspondence Address: KLARQUIST SPARKMAN, LLP, 121 SW SALMON STREET, SUITE 1600, PORTLAND, OR, 97204, US

(21) Appl. No.: 10/579,428

(22) PCT Filed: Jan. 21, 2005
(86) PCT No.: PCT/EP05/00695

§ 371 Date: Dec. 6, 2006

(30) Foreign Application Priority Data
Jan. 21, 2004 (ES) P200400120

(51) Int. Cl. C12P 21/06; C12N 7/00; C12N 5/06; C12N 15/86; C07K 14/03

(52) U.S. Cl. 435/69.1; 435/456; 435/348; 435/235.1; 977/802; 530/350

(57) ABSTRACT

The chimeric empty capsids of the infectious bursal disease virus (IBDV) are constituted by assembly of (i) IBDV pVP2 proteins and (ii) fusion proteins comprising a region A constituted by the IBDV VP3 protein bound to a region B constituted by a heterologous polypeptide comprising a polypeptide of interest, such as a polypeptide useful in vaccination, therapy or diagnosis.

### FIELD OF THE INVENTION

The invention is related to the production of chimeric empty particles of the infectious bursal disease virus (IBDV) and their applications.

### BACKGROUND OF THE INVENTION

Viral particles are structures specialized in the packaging and incorporating in vehicles of nucleic acids and proteins. A general feature of viral particles is their excellent ability for the immune response stimulation of the host These properties make viral particles agents of extraordinary interest for the development both of intracellular delivery systems and for the generation of particulate vaccines. The use of different genetic expression systems has facilitated the production of viral-like particles or empty viral capsids (VLPs) of different types of viruses (U.S. Pat. No. 6,458,362 Casal, et al. 2002. Recombinant VP2 parvoviral pseudo-particles encoding CTL or T-helper cell epitopes; U.S. Pat. No. 5,932,426 Baralle, et al. 1999. Molecular presenting system; U.S. Pat. No. 6,602,705 Barnett, et al. 2003 Expression of HIV polypeptides and production of virus-like particles). The genetic manipulation of these expression systems in turn allows the production of VLPs containing heterologous amino acid sequences coming from proteins other than those forming the native viral capsid. These VLPs are generically called heterotypical, recombinant or chimeric VLPs (CVLPs). CVLPs have mainly been used for two purposes: (i) generation of multivalent vaccines by means of immunologically relevant heterologous peptides (Kingsman, A. J., N. R. Burns, G. T. Layton, and S. E. Adams. 1995. Yeast retrotransposon particles as antigen delivery systems. Ann. N. Y. Acad. Sci. 754: 202-213; Lo-Man, R., P. Rueda, C. Sedlik, E. Deriaud, I. Casal, and C. Leclerc. 1998. A recombinant virus-like particle system derived from parvovirus as an efficient antigen carrier to elicit a polarized Th1 immune response without adjuvant. Eur. J. Immunol. 28: 1401-1407; Qiu, Z., D. Ou, H. Wu, T. C. Hobma, and S. Gillam. 1994. Expression and characterization of virus-like particles containing rubella virus structural proteins. J. Virol. 68: 4086-4091); and (ii) modification of the tropism by means of insertion of amino acid sequences involved in interactions with receptor-ligand (Schmidt, U., Rudolf, R, and Bömh, G. 2001. Binding of external ligands onto an engineered virus capsid. Prot. Eng. 14: 769-774; Shin, Y. C., and Folk, W. R. 2003. Formation of polyoma virus-like particles with different VPI molecules that bind the urokinase plasminogen activator receptor. J. Virol. 77: 11491-11498).

CVLPs are generally obtained by means of the expression of the viral protein(s) responsible for the formation of the viral capsid, fused to the region encoding the polypeptide of interest.

The infectious bursal disease virus (IBDV), belonging to the Birnaviridae family, infects different bird species and is directly responsible for a severe immunosuppressive disease causing important economic losses in the world poultry industry.

IBDV particles are icosahedral, with T=13 symmetry, they lack an envelope and are formed by a single protein layer. Up until now, the approaches aimed at obtaining an atomic model for IBDV particles have failed. As a result, the structural information available is based on three-dimensional models generated from images obtained by electron cryomicroscopy of the purified virus and of the VLPs. Based on these studies, it has been verified that the outer surface of the particle is formed by a continuous lattice of 260 trimers of the VP2 protein (37 kDa) organized in five different formations. The inner face of the particles contains 200 trimers of the VP3 protein (29 kDa), the latter, independent from one another, are bound to the basal area of the VP2 trimers. It has been suggested that a third polypeptide, VP4 (28 kDa), could also be part of the particles, being located at the base of the pentamers forming the vertices of the icosahedral structure.

The VP2, VP3 and VP4 polypeptides are produced from the proteolytic processing of a polypeptide precursor of a size of 109 kDa This precursor is auto-catalytically processed, releasing the pVP2 (48 kDa), VP3 and VP4 polypeptides. The VP4 domain, which is located in the central region of the polyprotein, belongs to the Lon protease family and is responsible for the proteolytic cleavage. The pVP2 and VP3 polypeptides are directly responsible for the capsid assembly. The pVP2 product undergoes a last cleavage at its C-terminal end before giving rise to the mature form of the protein, VP2, which is the one found in purified particles (Da Costa, B., Chevalier, C., Henry, C., Huet, J. C., Petit, S., Lepault, J., Boot, H. & Delmas, B. (2002). The capsid of infectious bursal disease virus contains several small peptides arising from the maturation process of pVP2. Journal of Virology 76:2393-2402). This pVP2 processing is necessary for the correct formation of the capsids and requires the presence of VP3, although the responsible protease has not yet been identified (Maraver, A., Oña, A., Abaitua, F., González, D., Clemente, R., Diaz-Ruiz, A., Caston, J. R, Pazos, F. & Rodriguez, J. F. (2003). The oligomerization domain of VP3, the scaffolding protein of infectious bursal disease virus, plays a critical role for capsid formation. Journal of Virology 77:6438-49).

In general terms, morphogenesis is a vital process for the viral cycle requiring successive steps associated to modifications in the polypeptide precursors. As a result, viruses have developed strategies allowing the sequential and correct interaction between each one of their components. One of these strategies, frequently used by icosahedral viruses, is the use of polypeptides coming from a single polyprotein as the base of their structural components. In these cases, the suitable proteolytic processing of said polyprotein plays a crucial role in the assembly process.

This concept for the assembly of IBDV capsids has been demonstrated in earlier work (Fernández-Arias, A., Risco, C., Martinez, S., Albar, J. P. & Rodriguez, J. F. (1998). Expression of ORF A1 of infectious bursal disease virus results in the formation of virus-like particles. Journal of General Virology 79, 1047-1054). Expression of the gene encoding for the IBDV polyprotein in eukaryotic cells gives rise to the formation of VLPs that are completely morphologically and biochemically indistinguishable from the IBDV virions. It has also been shown that the assembly of the capsids requires only the synthesis and correct processing of the viral polyprotein and is independent of the presence of the viral genome or of other proteins encoded by the viral genome, such as VP5 and VP1.

The results obtained to date from the IBDV gene expression in different recombinant systems has allowed concluding that: i) the assembly process is independent of the presence of genetic material of the virus, ii) only the polypeptides encoded by the polyprotein gene are necessary for the assembly, and iii) the assembly requires a coordinated interaction between the pVP2 and VP3 polypeptides.

However, it must be indicated that it is not known if the VP2/VP3 interaction is established between VP2 and VP3 domains of the polyprotein precursor when it has not yet undergone modifications, or on the contrary, if this interaction occurs after the processing of the precursor. Furthermore, current information does not exclude the possibility that VP4 could play a relevant role in the morphogenesis of the viral capsid. In fact, IBDV VLPs formed by assembly of the IBDV VP2, VP3 and VP4 proteins have been disclosed (U.S. Pat. No. 6,528,063, U.S. Pat. No. 5,788,970 and JP 5194597).

The work developed by the same inventors has enabled establishing systems for obtaining IBDV VLPs using different eukaryotic expression vectors. These vectors have been used for EBDV polyprotein expression in the absence or presence of the viral VP1 RNA polymerase. The biochemical characterization of purified VLPs demonstrates that they contain pVP2, VP2 and VP3 proteins when only the viral polyprotein is expressed, and the pVP2, VP2, VP3 and VPI proteins when the simultaneous expression of the polyprotein and viral RNA polymerase is carried out (Fernández-Arias, A., Risco, C., Martinez, S., Albar, J. P. & Rodriguez, J. F. (1998). Expression of ORF A1 of infectious bursal disease virus results in the formation of virus-like particles. Journal of General Virology 79: 1047-1054; Martinez-Torrecuadrada, J. L., Castón, J. R., Castro, M., Carrascosa, J. L., Rodriguez, J. F. & Casal, J. I. (2000). Different architectures in the assembly of infectious bursal disease virus capsid proteins expressed in insect cells. Virology 278: 322-331; Maraver, A., et al., (2003) cited supra; Lombardo, E., Maraver, A., Castón, J. R., Rivera, J., Fernández-Arias, A., Serrano, A., Carrascosa, J. L. & Rodriguez, J. P. (1999). VP1, the putative RNA-dependent RNA polymerase of infectious bursal disease virus, forms complexes with the capsid protein VP3, leading to efficient encapsidation into virus-like particles. Journal of Virology 73: 6973-6983).

On the other hand, patent application WO 02/088339 discloses IBDV viral-like particles formed by assembly of chimeric proteins comprising the IBDV polyprotein bound at its carboxyl terminal end to a polypeptide.

However, CVLPs solely based on IBDV pVP2 and VP3, the latter VP3 protein being fused to a polypeptide of interest, or their potential as vehicles of products of interest, have not been previously disclosed.

### SUMMARY OF THE INVENTION

The invention is faced with the problem of providing new tools for incorporating in vectors or vehicles products of interest, such as molecules with biological activity, for example drugs, polypeptides, proteins, nucleic acids, etc.

The solution provided by this invention is based on it being possible to generate, based on the simultaneous expression of the IBDV pVP2 and VP3 proteins, the latter genetically modified to include a nucleotide sequence encoding for a heterologous polypeptide comprising a polypeptide of interest, IBDV chimeric empty capsids (CVLPs). The resulting CVLPs are formed by assembly of (i) IBDV pVP2 proteins and (ii) fusion proteins comprising a region A constituted by the IBDV VP3 protein bound to a region B constituted by a heterologous polypeptide comprising a polypeptide of interest, wherein said region B is bound to the amino- or carboxy-terminal end of said IBDV VP3 protein. These CVLPs can be used for therapeutic, preventive or diagnostic purposes, etc., for example in the manufacture of gene therapy vectors or vaccines.

The inventors had previously found that when IBDV VPX (pVP2) and VP3 proteins are expressed from independent genes, empty IBDV particles (VLPs) are formed. These VLPs are structurally identical to those obtained by means of expression of the ORF corresponding to the E3DV polyprotein. As part of the development of new vaccination strategies, the possibility of using this IBDV VLP production strategy for obtaining CVLPs which contained heterologous amino acid sequences, corresponding to peptides of interest, such as a histidine tag (Example 1), GFP (Example 2) and finally peptides involved in immune response induction (Example 3), was analyzed. As is demonstrated, the fusion of heterologous sequences in these constructs is not an obstacle for the formation of CVLPs.

As a study model of peptide-transporting CVLPs involved in an immune response, the possibility of obtaining CVLPs which had the sequence corresponding to the CD8 epitope (E-CD8) of the malaria CS protein (Plasmodium yoelii) was approached. This epitope is responsible for the CD8-specific cellular immune response induction against this pathogen, which can be quantified by means of the ELISPOT technique in splenocyte cultures from BALB/c mice (Example 3).

In summary, the obtained results clearly show that: (i) the expression system used allows obtaining IBDV CVLPs containing heterologous amino acid sequences; and (ii) immunization with said IBDV CVLPs induces a specific immune response to the heterologous amino acid sequence present in the CVLPs.

Therefore, an aspect of the present invention is related to an IBDV chimeric empty capsid characterized in that it is constituted by assembly of (i) IBDV pVP2 proteins and (ii) fusion proteins comprising a region A constituted by the IBDV VP3 protein bound to a region B constituted by a heterologous polypeptide comprising a polypeptide of interest.

A further aspect of this invention is related to a process for producing said IBDV CVLPs provided by this invention, based on the gene coexpression of said IBDV pVP2 and fusion proteins as two independent genes.

The nucleic acids, gene constructs, expression systems and host cells developed for implementing said process of producing said IBDV CVLPs, as well as their use for the production of said IBDV CVLPs, constitute further aspects of the present invention.

Said IBDV CVLPs have the ability to incorporate in vectors or vehicles products of interest such as molecules with biological activity, for example, polypeptides, proteins, nucleic acids, etc. In a particular embodiment, said IBDV CVLPs can internally incorporate in vehicles antigens and immune response inducers in animals or humans to whom it is supplied, whereby they can be used in the manufacture of vaccines against human and animal diseases caused by viruses, bacteria, parasites or any other type of microorganism or against tumor diseases. In another particular embodiment, said IBDV CVLPs are used in the manufacture of gene therapy vectors.

Therefore, in a further aspect, the present invention is related to the use of said IBDV CVLPs in the manufacture of medicaments, such as vaccines and gene therapy vectors. Said vaccines and vectors constitute further aspects of the present invention.

### BRIEF DESCRIPTION OF THE FIGURES

FIG. 1. (a) The diagram schematizes the proteolytic processing steps necessary for the formation of mature VP2 and VP3 capsid proteins from the polyprotein precursor. (b) The diagram reflects different genetic constructs derived from the IBDV polyprotein described up until now, as well as the structures produced by means of its expression in different heterologous systems. The numbers indicate the position corresponding to the first and last amino acid residue of the polyprotein present in each one of the constructs. The lower portion of the figure shows images obtained by means of transmission electron microscopy of the structures obtained by means of expression of the different constructs. The bar corresponds to 50 nm. The data has been taken from the following literature references: Fernández-Arias et al., (1998), cited supra; Maraver et al., (2003), cited supra; Martinez-Torrecuadrada et al., (2000), cited supra; Castón et al., 2001. C terminus of infectious bursal disease virus major capsid protein VP2 is involved in definition of the t number for capsid assembly. Journal of Virology 75, 10815-10828.

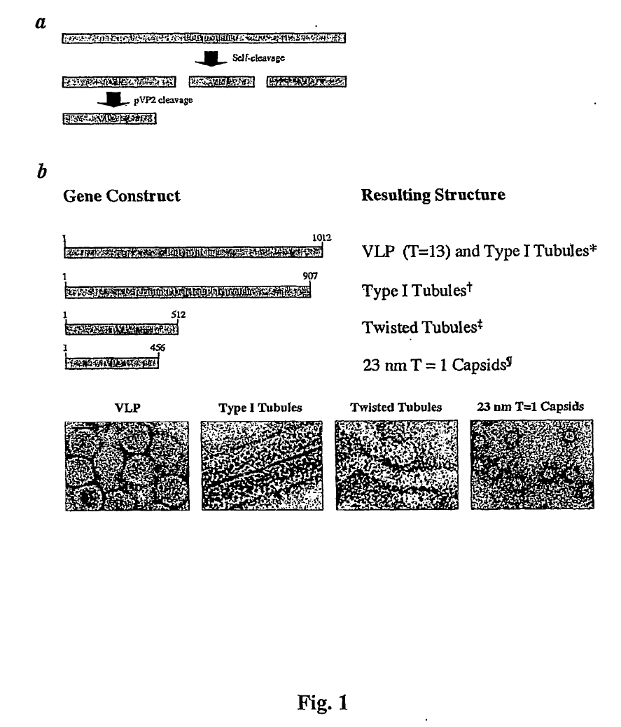

FIG. 2. Microscopic analysis of H5 insect cells coexpressing pVP2 and VP3. The pVP2 and VP3 protein subcellular distribution was analyzed by means of confocal immunomicroscopy. Cells infected with the FB/pVP2 (a), FB/VP3 (b), or FBD/pVP2-VP3 (c-e) rBVs were incubated with rabbit anti-pVP2 serum and rat anti-VP3 serum. Then the cells were incubated with goat anti-rabbit IgG serum coupled to Alexa 488 (red) and goat anti-rat IgG serum coupled to Alexa 594 (green). The cores were stained with To-Pro 3 (blue). (e) Overlaying of the images shown in panels (c) and (d). Electron microscopy images corresponding to sections of H5 cells infected with different genetic constructs derived from the IBDV polyprotein. (f) Low-magnification image of an H5 cell infected with a parental Fb virus. The insert corresponds to an enlarged detail of the area indicated by the box. (g) Low-magnification image of an H5 cell infected with the FBD/pVPX-VP3 virus. The insert corresponds to an enlarged detail of the area indicated by the box. (h) High-magnification image of an H5 cell infected with the FBD/pVPX-VP3 virus showing the formation of IBDV structures in detail. (i) High-magnification image of a BSC1 cell infected with the VTLacOI/POLY recombinant vaccine virus showing structures similar to those detected in panel (h). The bars indicate 600 nm (panels f and g) and 200 nm (panels h and i).

FIG. 3. Structural and biochemical characterization of the structures derived from IBDV produced in insect cells coinfected with the FB/pVP2 +FB/his-VP3 recombinant baculoviruses (rBV). Cells coinfected with FB/pVP2 and FB/his-VP3 rBVs, or infected with the FBD)/Poly-VP1 or FB/pVP2 virus, were used to purify structures derived from IBDV by means of centrifugation on sucrose gradients. Panels (a), (b), and (c) show transmission electron microscopy images corresponding to fraction 4 of the gradients obtained from infections with FBD/Poly-VPI, FB/pVP2+FB/his-VP3, and FB/pVP2, respectively. Panel (d) shows the results of a Western blot analysis of the sucrose gradients corresponding to the cultures infected with FBD/Poly-VP1 and FB/pVP2+FB/his-VP3, respectively. The total extracts (input) and the different fractions of the sucrose gradients (fraction F1 corresponds to the bottom of the gradient) were analyzed by means of Western blot using specific sera against the IBDV VP1, pVP2, VP3, and VP4 proteins, respectively. The molecular mass of the immunoreactive polypeptides is indicated in kDa.

FIG. 4. Biochemical and structural characterization of IBDV VLPs produced in S. cerevisiae transformed with the plasmid pESCURA/pVP2-VP3-GFP. A S. cerevisiae culture transformed with the plasmid pESCURA/pVP2-VP3-GFP was grown at 30° C. in a medium supplemented with the inducer galactose. At 18 hours, the culture was harvested and centrifuged. The resulting sediment was processed by means of fractioning in a 25-50% linear sucrose gradient. A) Biochemical analysis of samples corresponding to the sediment before fractioning (T) as well as the different fractions of the sucrose gradient. The samples were analyzed by means of SDS-PAGE and Western blot using specific antibodies against VP3 (anti-VP3) and pVP2 (anti-pVP2) proteins. The arrows indicate the positions of the immunoreactive bands corresponding to the VP3-GFP (61 kDa) and pVP2 (48 kDa) proteins, respectively. B) The structural analysis of the obtained samples was carried out by means of TEM. The image corresponds to a micrography obtained from an aliquot corresponding to the mixture of fractions 7, 8 and 9 of the sucrose gradient. The sample was stained with uranyl acetate and observed by means of TPM. The bar corresponds to 65 nm. C) VLPs sample obtained by means of the IBDV polyprotein expression in mammal cells by means of infection with the VT7/Poly recombinant vaccine virus (Fernández-Arias et al., (1998), cited supra). The bar corresponds to 65 nm.

FIG. 5. Structural and biochemical characterization of QVLPs-CD8. Panel A shows a TEM image of a sample stained with uranyl acetate corresponding to fraction 4 of a sucrose gradient used for the purification of structures carried out on an insect cell extract coinfected with the FB/pVP2 and PF/his-CD8-VP3 rBVs. The bar indicates 100 nm. Panel B shows the SDS-PAGE and Western blot analyses carried out with an antibody against VP3 protein, of a sample corresponding to fraction 4 (QVLPs-CD8) of a sucrose gradient used for the purification of structures carried out on an insect cell extract co-infected with the FB/pVP2 and PF/his-CD8-VP3 rBVs (see panel A). A sample of purified IBDV virus (IBDV) was used as a control. The sizes of the molecular mass (MW) markers, as well as the molecular mass estimated for the VP3 and his-CD8-VP3 proteins, were indicated.

FIG. 6. Enhancing effect of the specific anti-malaria CD8 cellular immune response by means of immunization with IBDV CVLPs containing the Plasmodium yoelii CDS epitope. Groups of 4 mice from the BALB/c strain were intraperitoneally inoculated with 50 μg/mouse of QVLPs-CD8 (group IV) or non-chimeric VLPs (group IIII). A group was inoculated with VVpJRCS (10⁷ pfu/mouse), a recombinant virus expression the whole Plasmodium yoelii CHITOSAN protein (group II) as a control. 15 days later, the mice of all the groups were intraperitoneally immunized with VVpJRCS (10⁷ pfu/mouse). One of the groups received at that time a single dose of the viral vector (group I). 15 days after the second immunization, the animals were sacrificed, the spleen was removed and the ELISPOT was carried out against the malaria CD8 peptide. Panel A shows the image of the ELISPOT wells carried out with different concentrations of splenocytes obtained from the mice belonging to each one of the groups after their incubation in the presence (+CD8 peptide) or absence (−CD8 peptide) of the CD8 peptide. Panel B shows a graph of the results obtained as a number of specific IFN-γ/10⁶ splenocyte secreting cells.

### DETAILED DESCRIPTION OF THE INVENTION

In a first aspect, the invention provides an chimeric empty capsid of the infectious bursal disease virus (IBDV), hereinafter CVLP of the invention, characterized in that it is constituted by assembly of (i) IBDV pVP2 proteins and (ii) fusion proteins comprising a region A constituted by the IBDV VP3 protein bound to a region B constituted by a heterologous polypeptide comprising a polypeptide of interest.

The term “IBDV”, as it is used in the present invention, refers to the different IBDV strains belonging to any of the known serotypes (1 or 2) [by way of illustration, see the review carried out by van den Berg T P, Eterradossi N, Toquin D, Meulemans G., en Rev Sci Tech 2000 19: 50943] and the terms “IBDV pVP2 protein” and “IBDV VP3 protein” refer to the different forms of the pVP2 and VP3 proteins representative of any of the mentioned IBDV strains [NCBI protein databank], according to the definition made by Sánchez and Rodriguez (1999) (Sánchez A B, Rodriguez J F. Proteolytic processing in infectious bursal disease virus: identification of the polyprotein cleavage sites by site-directed mutagenesis. Virology. 1999 Sep. 15; 262(1):190-199), as well as proteins substantially homologous to said IBDV pVP2 and VP3 proteins, i.e. proteins the amino acid sequences of which have a degree of identity regarding said IBDV pVP2 and VP3 proteins of at least 60%, preferably of at least 80%, more preferably of at least 90% and even more preferably of at least 95%.

The IBDV pVP2 protein present in the CVLP of the invention can be any pVP2 protein representative of any IBDV strain, for example, the full-length pVP2 protein of IBDV Soroa strain [NCBI, access number AAD30136].

The fusion protein present in the CVLP of the invention comprises a region A constituted by the IBDV VP3 protein bound to a region B constituted by a heterologous polypeptide comprising a polypeptide of interest. In a particular embodiment, said region B is bound to the amino-terminal region or to the carboxy-terminal region of said IBDV VP3 protein.

The IBDV VP3 protein, constituting region A of said fusion protein, can be any VP3 protein representative of any IBDV strain, for example, the full-length VP3 protein of IBDV Soroa strain [NCBI, access number AAD30136].

Region B present in said fusion protein is constituted by a heterologous polypeptide comprising a polypeptide of interest. As it is used in the present invention, the term “heterologous polypeptide” refers to a polypeptide not belonging to the native IBDV capsid. The size of the polypeptide of interest can vary within a broad interval, from a few amino acids up to hundreds of amino acids. Said polypeptide of interest can be virtually any polypeptide, regardless of its origin (eukaryotic, prokaryotic, viral, etc.), susceptible to being expressed in a recombinant manner. However, in a particular embodiment said polypeptide of interest is a polypeptide useful in vaccination, therapy or diagnosis, such as an epitope or determining antigen capable of inducing an immune response in animals and humans against diseases caused by viruses, bacteria, parasites or any other type of microorganism, or against tumor diseases.

In a particular embodiment, said region B comprises a single polypeptide of interest. However, in another particular embodiment, said region B comprises two or more polypeptides of interest, equal or different, which can be forming tandems.

In a particular embodiment, said fusion protein comprises a region A bound to a single region B. In this case, said region B can be bound to the amino-terminal region of VP3, or alternatively to the carboxy-terminal region of VP3, present in region A. As previously mentioned region B can contain one or more polypeptides of interest. In a particular embodiment, said region B contains a single polypeptide of interest, whereas in another particular embodiment, said region B comprises two or more different polypeptides of interest.

In another particular embodiment, said fusion protein comprises a region A bound to two regions B, one of them bound to the amino-terminal region of VP3 present in region A and the other one to the carboxy-terminal region of VP3 present in region A. Said regions B can be equal or different, and each one of them can contain one or more polypeptides of interest, which can be equal to or different from one another. In a specific embodiment, the fusion protein comprises a region A bound to a first region B containing a first polypeptide of interest (B1) and a second region B containing a second polypeptide of interest (B2). Said polypeptides of interest (B1) and (B2) can be equal or different. In a specific embodiment, said polypeptides of interest (B1) and (B2) are different from one another.

Generally, region A (constituted by the IBDV VP3 protein) is not bound directly to said region B (constituted by the heterologous polypeptide comprising a polypeptide of interest), but rather through a linker polypeptide between said regions A and B. Therefore, if desired the fusion protein of the invention can further contain a linker polypeptide located between said regions A and B. Advantageously, said linker polypeptide is a peptide with structural flexibility, preferably a polypeptide giving rise to a non-structured domain able to induce an immune response or not. By way of illustration, said flexible peptide can contain repetitions of amino acid residues, particular Gly and Ser residues, or any other suitable repetition of amino acid residues.

The CVLPs of the invention can be obtained by means of the simultaneous expression of said IBDV pVP2 proteins and said fusion protein comprising said regions A and B, in suitable host cells. Said suitable host cells are cells containing the encoding nucleotide sequence of said fusion protein comprising regions A and B, and the encoding nucleotide sequence of the EBDV pVP2 protein, either in a single gene construct or in two gene constructs. In a particular embodiment, said suitable host cells are cells that are transformed, transfected or infected with a suitable expression system, such as an expression system comprising a gene construct, wherein said gene construct comprises the nucleotide sequence encoding for said fusion protein comprising regions A and B, and the nucleotide sequence encoding for the IBDV pVP2 protein, or else alternatively with an expression system comprising a first gene construct comprising the nucleotide sequence encoding for said fusion protein comprising regions A and B, and a second gene construct comprising the nucleotide sequence encoding for the IBDV pVP2 protein.

Therefore, in another aspect, the invention provides a nucleic acid, the nucleotide sequence of which comprises the nucleotide sequence encoding for said fusion protein forming part of the CVLPs of the invention and comprising a region A constituted by the IBDV VP3 protein bound to a region B constituted by a heterologous polypeptide comprising a polypeptide of interest, wherein said regions B bound to the amino-terminal region or to the carboxy-terminal region of said IBDV VP3 protein. Optionally, the nucleic acid provided by this invention can contain the nucleotide sequence encoding for IBDV pVP2 if desired. More specifically, in a particular embodiment, the nucleic acid sequence provided by this invention comprises (i) a nucleotide sequence comprising the open reading frame or encoding region corresponding to the IBDV VP3 protein and (ii) a nucleotide sequence comprising the open reading frame or encoding region of one or more heterologous polypeptides comprising one or more polypeptides of interest, and optionally if desired, (iii) a nucleotide sequence comprising the open reading frame or encoding regions corresponding to the IBDV pVP2 protein.

In another particular embodiment, the nucleic acid sequence provided by this invention comprises (i) a nucleotide sequence comprising the open reading frame or encoding region corresponding to the IBDV VP3 protein, (ii) a first nucleotide sequence comprising the open reading frame or encoding region of one or more heterologous polypeptides comprising the open reading frame or encoding region of one or more heterologous polypeptides comprising one or more polypeptides of interest, (ii′) a second nucleotide sequence comprising the open reading frame or encoding region of one or more heterologous polypeptides comprising one or more polypeptides of interest, wherein said nucleotide sequence can be equal to or different from each first nucleotide sequence, and optionally if desired, (iii) a nucleotide sequence comprising the open reading frame or encoding region corresponding to the IBDV pVP2 protein In this case, one of said first or second nucleotide sequence is operatively bound to 5′ end of the nucleotide sequence comprising the open reading frame or encoding region corresponding to the EBDV VP3 protein and the other one is operatively bound to the 3′ end of the nucleotide sequence comprising the open reading frame or encoding region corresponding to the IBDV VP3 protein.

As it is used in this description, the term “open reading frame corresponding to the pVP2 protein” or “open reading frame corresponding to the IDV VP3 proteins” includes, apart from the nucleotide sequences of said open reading frames, other open reading frames analogous to the same encoding frames of the IBDV pVP2 and VP3 proteins. Likewise, the term “open reading frame of one or more heterologous polypeptides comprising one or more polypeptides”, includes any encoding nucleotide sequence of said heterologous polypeptide(s) comprising one or more polypeptides of interest. As it is used herein, the term “analogous” intends to include any nucleotide sequence which can be isolated or constructed on the base of the encoding nucleotide sequence of IBDV pVP2 and VP3, for example by means of the introduction of conservative or non-conservative nucleotide replacements, including the insertion of one or more nucleotides, the addition of one or more nucleotides at any of the ends of the molecule, or the deletion of one or more nucleotides at any end or inside of the sequence. Generally, a nucleotide sequence analogous to another nucleotide sequence is substantially homologous to said nucleotide sequence. In the sense used in this description, the expression “substantially homologous” means that at the nucleotide level, the nucleotide sequences in question have a degree of identity of at least 60%, preferably of at least 80%, more preferably of at least 90%, and even more preferably of at least 95%.

In another aspect, the invention provides a gene construct comprising a nucleic acid provided by this invention, i.e. a nucleic acid the nucleotide sequence of which comprises the nucleotide sequence encoding for said fusion protein comprising regions A and B, and optionally the nucleotide sequence encoding for said IBDV pVP2 protein. More specifically, in a particular embodiment, the gene construct provided by this invention comprises a nucleotide sequence comprising (i) a nucleotide sequence comprising the open reading frame or encoding region corresponding to the IBDV VP3 protein and (ii) a nucleotide sequence comprising the open reading frame or encoding region of one or more heterologous polypeptides comprising one or more polypeptides of interest, and optionally if desired, (iii) a nucleotide sequence comprising the open reading frame or encoding region corresponding to the IBDV pVP2 protein. In another particular embodiment, the gene construct provided by this invention comprises a nucleotide sequence comprising (i) a nucleotide sequence comprising (i) a nucleotide sequence comprising the open reading frame or encoding region corresponding to the IBDV VP3 protein, (ii) a first nucleotide sequence comprising the open reading frame or encoding region of one or more heterologous polypeptides comprising one or more polypeptides of interest, (ii′) a second nucleotide sequence comprising the open reading frame or encoding region of one or more heterologous polypeptides comprising one or more polypeptides of interest, wherein said second nucleotide sequence can be equal to or different from said first nucleotide sequence, and optionally if desired (iii) a nucleotide sequence comprising the open reading frame or encoding region corresponding to the IBDV pVP2 protein. In this case, one of said first or second nucleotide sequences is operatively bound to the 5′ end of the nucleotide sequence comprising the open reading frame or encoding region corresponding to the IBDV VP3 protein, and the other one is operatively bound to the 3′ end of the nucleotide sequence comprising the open reading frame or encoding region corresponding to the IBDV VP3 protein.

In another aspect, the invention provides an expression vector or system selected from:
a) an expression system comprising a gene construct provided by this invention, operatively bound to transcription, and optionally translation, control elements, wherein said gene construct comprises the nucleotide sequence encoding for said fusion protein comprising regions A and B and the nucleotide sequence encoding for the IBDV pVP2 protein; and
b) an expression system comprising a first gene construct provided by this invention, operatively bound to transcription, and optionally translation, control elements, wherein said first construct comprises the nucleotide sequence encoding for said fusion protein comprising regions A and B, and a second gene construct operatively bound to transcription, and optionally translation, control elements, comprising the nucleotide sequence encoding for the IBDV pVP2 protein.

In a particular embodiment, the expression system provided by this invention comprises a gene construct comprising (i) a nucleotide sequence comprising the open reading frame or encoding region corresponding to the IBDV VP3, (ii) a nucleotide sequence comprising the open reading frame or encoding region of one or more heterologous polypeptides comprising one or more polypeptides of interest, and (iii) a nucleotide sequence comprising the open reading frame or encoding region corresponding to the IBDV pVP2 protein, wherein said gene construct is operatively bound to transcription, and optionally translation, control elements.

In another particular embodiment, the expression system provided by this invention comprises a first gene construct, operatively bound to transcription, and optionally translation, control elements, said first gene construct comprising (i) a nucleotide sequence comprising the open reading frame or encoding region corresponding to the IBDV VP3 protein, and (ii) a nucleotide sequence comprising the open reading frame or encoding region of one or more heterologous polypeptides comprising one or more polypeptides of interest, and a second gene construct, operatively bound to transcription, and optionally translation, control elements, said second gene construct comprising a nucleotide sequence comprising the open reading frame or encoding region corresponding to the IBDV pVP2 protein The transcription, and optionally translation, control elements present in the expression system provided by this invention include promoters, directing the transcription of the nucleotide sequences of interest (pVP2, VP3 and heterologous polypeptide) to which it is operatively linked, and other sequences necessary or suitable for the transcription and its suitable regulation in time and place, for example, start and end signals, cleavage sites, polyadenylation signal, replication origin, transcriptional activators (enhancers), transcriptional silencers (silencers), etc.

Virtually any suitable expression system or vector can be used in the generation of the expression system provided by this invention. By way of illustration, said suitable expression or vector systems can be selected, according to the conditions and needs of each specific case, from plasmids, bacmids, yeast artificial chromosomes (YACs), bacteria artificial chromosomes (BACs), bacteriophage P1-based artificial chromosomes (PACs), cosmids, or viruses, which can further have a heterologous replication origin, for example, bacterial or of yeast, so that it may be amplified in bacteria or yeasts, as well as a marker usable for selecting the transfected cells different from the gene or genes of interest. These expression systems or vectors can be obtained by conventional methods known by persons skilled in the art [Sambrook, J., Fritsch, E. F., and Maniatis, T. (1989). Molecular cloning: a laboratory manual, 2nd ed. Cold Spring Harbor Laboratory] and form part of the present invention. In a particular embodiment, said expression or vector system is a plasmid, such as a plasmid suitable for transforming yeasts, for example, the plasmid called pESCURA/pVP2-VP3-GFP (Example 2), or a virus, such as a recombinant baculovirus (rBV), for example, the rBV called FBD/pVP2-his-VP3 (Example 1.2), simultaneously expressing both proteins (IBDV pVP² and his-VP3) in insect cells during the replication cycle, or the rBVs called FB/pVP2 and FB/his-VP3 (Example 1.1) expressing the IBDV pVP2 and his-VP3.proteins, respectively, when coinfecting insect cells, obtaining IBDV CVLPs with the six histidine (6 his) heterologous polypeptide, or the rBVs called FB/pVP2 and FB/his-CD8-VP3 (Example 3) expressing IBDV pVP2 proteins and his-CD8-VP3, respectively, when co-infecting insect cells, forming the capsids called CD8-CVLPs.

In another aspect, the invention provides a host cell containing the encoding nucleotide sequence of said fusion protein comprising regions A and B, and the encoding nucleotide sequence of the IBDV pVP2 protein, either in a single gene construct or in two different gene constructs. In a particular embodiment, said host cell is a host cell that is transformed, transfected or infected with (i) an expression system provided by this invention comprising either a gene construct wherein said gene construct comprises the nucleotide sequence encoding for said IBDV pVP2 protein and the nucleotide sequence encoding for said fusion protein comprising regions A and B, and the encoding nucleotide sequence of the IBDV pVP2 protein, or else alternatively with (ii) an expression system comprising a first construct comprising the nucleotide sequence encoding for said fusion protein comprising regions A and B, and second gene construct comprising the nucleotide sequence encoding for said IBDV pVP2 protein.

In a particular embodiment, the host cell provided by this invention is a host cell that is transformed, transfected or infected with an expression system comprising a gene construct comprising (i) a nucleotide sequence comprising the open reading frame or encoding region corresponding to the IBDV VP3 protein, (ii) a nucleotide sequence comprising the open reading frame or encoding region of a heterologous polypeptide comprising a polypeptide of interest, and (iii) a nucleotide sequence comprising the open reading frame or encoding region corresponding to the IBDY pVP2 protein, wherein said gene construct is operatively bound to transcription, and optionally translation, control elements.

In another particular embodiment, the host cell provided by this invention is a host cell that is transformed, transfected or infected with (a) a first gene construct, operatively bound to transcription, and optionally translation, control elements, said first gene construct comprising (i) a nucleotide sequence comprising the open reading frame or encoding region corresponding to the IBDV VP3 protein, and (ii) a nucleotide sequence comprising the open reading frame or encoding region of a heterologous polypeptide comprising a polypeptide of interest, and with (b) a second gene construct, operatively bound to transcription, and optionally translation, control elements, said second gene construct comprising a nucleotide sequence comprising the open reading frame or encoding region corresponding to the IBDV pVP2 protein.

Virtually any host cell susceptible to being transformed, transfected or infected by an expression system provided by this invention can be used, for example, mammal cells, bird cells, insect cells, yeasts, etc; however, in a particular embodiment, said host cell is selected from yeasts and insect cells. Yeasts are suitable due to the simplicity and production cost. Insect cells are suitable when the expression system comprises one or two recombinant baculoviruses (rBV). The use of rBV is advantageous due to biosafety issues related to the host range of the baculoviruses, incapable of replicating in other cell types which are not insect cells.

In a particular embodiment, the invention provides a host cell, such as a yeast, for example, Saccharomyces cerevisiae, Saccharomyces pombe, etc., transformed with an expression system, such as a plasmid or an expression vector, comprising a gene construct provided by this invention comprising the nucleotide sequence encoding for said fusion protein comprising regions A and B, and the nucleotide sequence encoding for the IBDV pVP2 protein.

In another particular embodiment, the invention provides a host cell, such as an insect cell, infected with an expression system, such as a recombinant baculovirus, comprising a gene construct provided by this invention comprising the nucleotide sequence encoding for said fusion protein comprising regions A and B, and the nucleotide sequence encoding for the IBDV pVP² protein.

In another particular embodiment, the invention provides a host cell, such as an insect cell, coinfected with an expression system comprising a first recombinant baculovirus comprising a gene construct provided by this invention comprising the nucleotide sequence encoding for said fusion protein comprising regions A and B, and with a second recombinant baculovirus comprising a gene construct provided by this invention comprising the nucleotide sequence encoding for the IBDV pVP2 protein.

In another aspect, the invention provides a process for the production of CVLPs of the invention comprising culturing a host cell provided by this invention containing the encoding nucleotide sequence of said fusion protein comprising regions A and B, and the encoding nucleotide sequence of IBDV pVP2, either in a single gene construct or in two different gene constructs, and simultaneously expressing said IBDV pVP2 proteins and fusion protein comprising regions A and B, and if desired, recovering said CVLPs of the invention. In a particular embodiment, said host cell provided by this invention is a cell that is transformed, transfected or infected with a suitable expression system, such as an expression system comprising a gene construct, wherein said gene construct comprises the nucleotide sequence encoding for said fusion protein comprising regions A and B, and the nucleotide sequence encoding for IBDV pVP2, or else alternatively with an expression system comprising a first gene construct comprising the nucleotide sequence encoding for said fusion protein comprising regions A and B, and a second gene construct comprising the nucleotide sequence encoding for IBDV pVP2.

Said process therefore comprises the gene coexpression of said IBDV pVP2 proteins and fusion proteins comprising regions A and B as two independent genes. After the simultaneous expression of said proteins (IBDV pVP2 and fusion proteins comprising regions A and B) in said cells, the expressed proteins are assembled and form the CVLPs of the invention, which can be isolated or withdrawn from the medium and purified if desired. The isolation and purification of said CVLPs of the invention can be carried out by conventional methods, for example, by means of fractioning on sucrose gradients.

In a particular embodiment, the simultaneous gene coexpression of IBDV pVP2 proteins and fusion proteins comprising regions A and B is carried out by means of the use of an rBV allowing the simultaneous expression of said proteins from two independent chimeric genes in insect cells. In this case, the process for the production of CVLPs of the invention provided by this invention comprises, first the obtainment of a gene expression system constituted by an rBV containing a gene construct simultaneously encoding for the IBDV pVP2 proteins and for said fusion proteins comprising regions A and B, such as the rBV called FBD/pVP2-his-VP3 (Example 1.2), or alternatively the obtainment of an rBV containing a gene construct encoding for the IBDV pVP2 protein and the obtainment of another rBV containing a gene construct encoding for said fusion protein comprising regions A and B, such as the rBVs called FB/pVP2 and FB/his-VP3 (Example 1.1), or rBVs called FB/pPV2 and FB/his-CD8-VP3 (Example 3), respectively, followed by the infection of insect cells with said expression system based on said recombinant baculovirus(es), expression of the recombinant proteins and if desired, isolation of the CVLPs of the invention formed by assembly of said IBDV pVP2 proteins and fusion proteins comprising regions A and B, and optionally subsequent purification of said CVLPs of the invention.

The construction of a recombinant baculovirus allowing the independent expression of the IBDV pVP2 proteins and the fusion proteins comprising regions A and B can be carried out by any person skilled in the art based on that described herein and on the state of the art concerning this technology (Cold Spring Harbor, N.Y.; Leusch M S, Lee S C, Olins P O. 1995. A novel host-vector system for direct selection of recombinant baculoviruses (bacmids) in Escherichia coli. Gene 160: 191-4; Luckow V A, Lee S C, Barry G F, Olins P O. 1993. Efficient generation of infectious recombinant baculoviruses by site-specific transposon-mediated insertion of foreign genes into a baculovirus genome propagated in Escherichia coli. J Virol 67: 4566-79).

In another particular embodiment, the gene coexpression of the IBDV pVP2 proteins and of the previously defined fusion proteins comprising regions A and B is carried out by means of the use of a vector allowing the expression of said proteins in yeast cells. In this case, the process for the production of CVLPs of the invention provided by this invention comprises, first, the obtainment of a gene expression system constituted by a plasmid containing a gene construct simultaneously encoding for the IBDV pVP2 proteins and for said fusion proteins comprising regions A and B, followed by the transformation of yeasts with said expression system, expression of the recombinant proteins, and if desired, isolation of the CVLPs of the invention formed by assembly of said IBDV pVP2 proteins and fusion proteins comprising regions A and B, and optionally subsequent purification of said CVLPs of the invention. In a specific embodiment, the suitable expression system for transforming yeasts is based on a pESC Yeast (Stratagene) expression system such as, for example, the plasmid pESCURA/pVP2NVP3-GFP (Example 2) containing a gene construct encoding for the IBDV pVP2 and VP3-GFP proteins.

The obtainment of yeasts transformed with a gene construct or with a suitable expression system or vector allowing the simultaneous expression of the IBDV pVP2 proteins and the fusion proteins comprising regions A and B can be carried out by any person skilled in the art based on that described herein and on the state of the art concerning this technology (PESC epitope tagging vectors Instructions manual. Stratagene www.stratagene.com; Sambrook, J., Fritsch, E. F., and Maniatis, T. (1989). Molecular cloning: a laboratory manual, 2nd ed. Cold Spring Harbor Laboratory).

In another aspect, the invention is related to the use of the gene expression system provided by this invention for producing and obtaining the CVLPs of the invention.

The CVLPs of the invention can be used as vectors or vehicles of products of interest, such as molecules with biological activity, for example, drugs, polypeptides, proteins, nucleic acids, etc., whereby they can be used for therapeutic or diagnostic or research purposes. In a particular embodiment, said molecules of biological interest include polypeptides of interest, such as antigens or immune response inducers in animals or humans to whom they are supplied, or nucleic acid sequences, useful in gene therapy, intended for being introduced inside the suitable cells.

Therefore, in another aspect, the invention is related to the use of the CVLPs of the invention in the manufacture of medicaments, for example vaccines, gene therapy vectors (delivery systems), etc. In a particular embodiment, said medicament is a vaccine intended for conferring protection against human or animal diseases caused by viruses, bacteria, parasites, or any other type of microorganism, or against tumor diseases. In another particular embodiment, said medicament is a gene therapy vector.

In another aspect, the invention provides a vaccine comprising a therapeutically effective amount of CVLPs of the invention, optionally together with one or more pharmaceutically acceptable adjuvants and/or vehicles. Said vaccine is useful for protecting animals and humans against diseases caused by microorganisms (viruses, bacteria, parasites, etc.), or against tumor diseases. In a particular embodiment, said vaccine is especially useful for simultaneously protecting animals or humans against the infection caused by two or more infectious disease-causing agents. By way of illustration, the vaccine provided by this invention can be used to protect birds, for example chickens, turkeys, geese, pheasants, quails, ostriches, etc., against the infectious bursal disease virus (IBDV) and against one or more infectious agents responsible for avian diseases (avian pathogens).

In the sense used in this description, the expression “therapeutically effective amount” refers to the amount of CVLPs of the invention calculated for producing the desired effect and will generally be determined, among others, by the characteristics of the CVLPs and the immunization effect to be achieved.

The pharmaceutically acceptable adjuvants and vehicles which can be used in said vaccines are those adjuvants and vehicles known by the persons skilled in the art and normally used in the manufacture of vaccines.

In a particular embodiment, said vaccine is prepared in form of an aqueous solution or suspension in a pharmaceutically acceptable diluent, such as saline solution, phosphate-buffered saline solution (PBS), or any other pharmaceutically acceptable diluent.

The vaccine provided by this invention can be administered by any suitable administration route which results in a protective immune response against the heterologous sequence or epitope used, to which end said vaccine will be formulated in the dosage form suited to the chosen administration route. In a particular embodiment, the administration of the vaccine provided by this invention is carried out parenterally, for example, intraperitoneally, subcutaneously, etc.

The following Examples illustrate the invention and should not be considered limiting of the scope thereof.

### EXAMPLE 1

### Obtaining IBDV CVLPs in Insect Cells

1.1 Obtaining IBDV CVLPs, VP2-his-VP3, by Means of Two Independent rBVs in Insect Cells

The results of a series of experiments designed to analyze the possibility of obtaining IBDV CVLPs from the coexpression of the EBDV pVP2 and VP3 proteins and a heterologous polypeptide from two independent chimeric genes are described in this example. To that end, two recombinant baculoviruses (rBVs) described above, FB/his-VP3 (Kochan, G., González, D. & Rodriguez, J. F. (2003). Characterization of the RNA binding activity of VP3, a major structural protein of IBDV. Archives of Virology 148, 723-744) and FB/VPX, herein cited as FB/pVP2, (Martinez-Torrecuadrada, J. L., Castón, J. R., Castro, M., Carrascosa, J. L., Rodriguez, J. F. & Casal, J. I. (2000). Different architectures in the assembly of infectious bursal disease virus capsid proteins expressed in insect cells. Virology 278, 322-331) have been used. These rBVs were generated by means of the cloning into suitable vectors of the complementary DNA (cDNA) encoders of the IBDV pVP2 and pVP3 proteins. Said cDNAs were obtained by RT-PCR from the A segment of the serotype I IBDV Soroa strain genome a (NCBI access number AAD30136). The rBV FB/his-VP3 expresses a chimeric VP3 protein which at its N-terminal end contains a tandem of six histidines fused to the VP3 sequence (Met754Glu1012 of the polyprotein) called his-VP3. rBV FB/pVP2 expresses the encoding region of the pVP2 protein (Met1-Ala512).

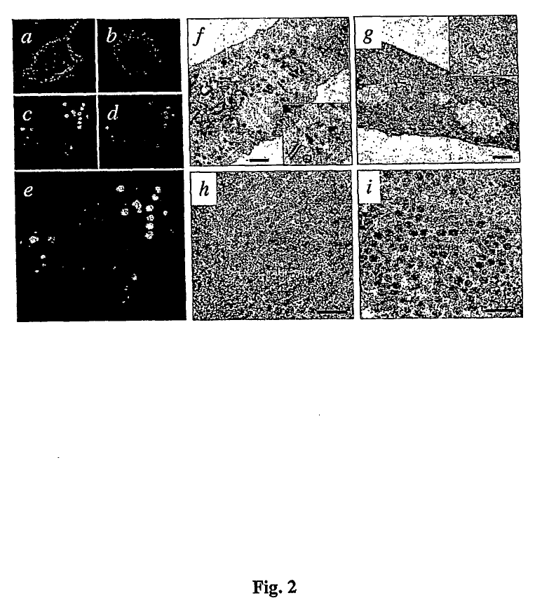

The analysis of the expression of these pVP2 and his-pVP3 proteins, whether independently or together, was carried out in cell cultures. To carry out these experiments, single layer cell cultures from the insect Trichloplusia ni (H5, Invitrogen) were used, which were grown on cover glasses. Said cultures were independently infected with FB/pVP2, FB/his-VP3, or coinfected with both rBVs. The multiplicity of infection was 5 pfu/cell. The cells were fixed at 48 hours post-infection h.p.i.), and incubated with rabbit anti-VP2 polyclonal serum and with rat anti-VP3 polyclonal serum (Fernández-Arias, A., Risco, C., Martinez, S., Albar, J. P. & Rodriguez, J. F. (1998). Expression of ORF A1 of infectious bursal disease virus results in the formation of virus-like particles. Journal of General Virology 79, 1047-1054). After successive washings, the cover glasses were incubated with goat anti-rat serum conjugated with Alexa 594 and goat anti-rabbit serum conjugated with Alexa 488 (Jackson Immunoresearch Laboratories, Inc.). The cellular cores were stained with the specific To-Pro-3 marker (Molecular Probes, Inc.). The samples were finally viewed by epifluorescence with a Zeiss Axiovert 200 microscope equipped with the Bio Rad Radiance 2100 confocal system. The images obtained were stored using the Laser Sharp Package (Bio Rad) software equipment As is shown in FIG. 2a, in the cultures infected with FB/pVP2, the anti-VP2 serum showed a fine granular signal mixed with tubular structures, both distributed throughout the cytoplasm. The anti-VP3 signal, detected in the cells infected with rBV FB/his-VP3, was characterized by the presence of spherical-shaped, and apparently hollow, accumulations around the core. In the cultures coinfected with both rBVs, a notable modification in the distribution pattern of both proteins was detected. In these cells, the specific signals of pVP2 and VP3 were collocated in spherical and dense accumulations, suggesting that their coexpression allowed the formation of pVP2/his-VP3 complexes (FIG. 2c to 2e).

For the purpose of characterizing these structures in greater detail, similar extracts corresponding to cells infected with FB/pVP2+FB/hisVP3 were analyzed by transmission electron microscopy (TEM). As a control, and in parallel, H5 cell cultures infected with the wild strain of the FBD (FastBacDual, Invitrogen) virus were analyzed by the same technique. After the infection, the cells were harvested after 48 hours, and processed as has been previously described (Fernández-Arias A, Risco C, Martinez S, Albar J P & Rodriguez J F. (1998). Expression of ORF A1 of infectious bursal disease virus results in the formation of virus-like particles. Journal of General Virology 79:1047-1054) for their analysis in ultrathin sections by TRANSMISSION ELECTRON MICROSCOPY. As is shown in FIG. 2, the cytoplasm of the coinfected cells contains aggregates formed by a mixture of tubules and structures similar to capsids (FIGS. 2g, 2h and 2i). These aggregates were not observed in any case in the samples corresponding to cells infected with wild FBD virus (FIG. 2f). The appearance and size of the tubules, as well as of the structures similar to capsids, was similar to those previously described in cell cultures infected with VT7/Poly, a recombinant of the vaccinia virus expressing the gene of the IBDV polyprotein (Fernández-Arias A, Risco C, Martinez S, Albar J P & Rodriguez J F. (1998). Expression of ORF A1 of infectious bursal disease virus results in the formation of virus-like particles. Journal of General Virology 79:1047-1054).

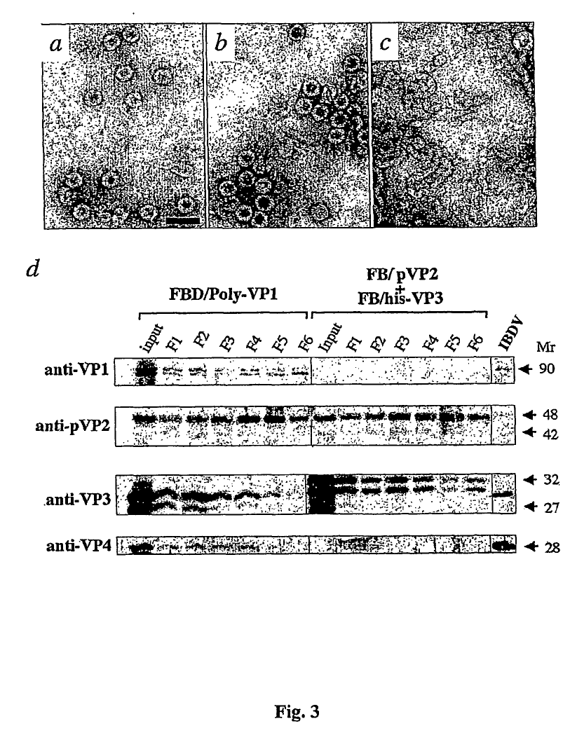

To unmistakably establish that the coexpression of pVP2 and his-VP3 enabled the assembly and, therefore, the obtainment of CVLPs, the decision was made to purify the formed particles. To that end, H5 cell cultures were infected with FB/pVP2+FB/his-VP3. At 60 h.p.i., the cells were homogenized and the extracts were separated on sucrose gradients as previously described (Lombardo E, Maraver A, Castón J R, Rivera J, Fernández-Arias A, Serrano A, Carrascosa J L & Rodriguez J F. (1999). VP1, the putative RNA-dependent RNA polymerase of infectious bursal disease virus, forms complexes with the capsid protein VP3, leading to efficient encapsidation into virus-like particles. Journal of Virology 73:6973-6983). After their centrifugation, the gradients were fractioned, and the different fractions were analyzed by TEM as previously described (Lombardo E, Maraver A, Castón J R, Rivera J, Fernández-Arias A, Serrano A, Carrascosa J L & Rodriguez J F. (1999). VP1, the putative RNA-dependent RNA polymerase of infectious bursal disease virus, forms complexes with the capsid protein VP3, leading to efficient encapsidation into virus-like particles. Journal of Virology 73:6973-6983). As a control, and subject to the same process, gradients corresponding to cell extracts infected with rBV FB/VPX or with rBV FBD/Poly-VPI, were fractioned. The recombinant virus FBD/Poly-VP1 simultaneously expresses the VP1 polypeptide and polyprotein. As was predictable, the infection with FBD/Poly-VP1 had a result of an efficient production of VLPs (Maraver A, Oña A, Abaitua F, González D, Clemente R, Diaz-Ruiz A, Castón J R, Pazos F & Rodriguez J F. (2003). The oligomerization domain of VP3, the scaffolding protein of infectious bursal disease virus, plays a critical role for capsid formation. Journal of Virology 77:6438-49). On the other hand, the fractions corresponding to the cells infected with FB/VPX only contain tubules of a twisted appearance. The gradients corresponding to cells coinfected with the rBVs FB/pVP2+FB/his-VP3 contain rigid type I tubules in the fractions near the bottom of the gradient, and CVLPs in the central and top fractions (FIG. 3b). The CVLPs isolated from the cells coinfected with rBV FB/pVP2+FB/his-VP3 had a diameter of 65-70 nm, as well as a typical polygonal contour, absolutely indistinguishable from the purified VLPs of cultures infected with FBD/Poly-VP1 (Maraver, A., Oña, A., Abaitua, F., González, D., Clemente, R., Diaz-Ruiz, A., Caston, J. R, Pazos, F. & Rodriguez, J. F. (2003). The oligomerization domain of VP3, the scaffolding protein of infectious bursal disease virus, plays a critical role for capsid formation. Journal of Virology 77:6438-49) or of the cultures infected with VT7/Poly (Fernández-Arias, A., Risco, C., Martinez, S., Albar, J. P. & Rodriguez, J. F. (1998). Expression of ORF A1 of infectious bursal disease virus results in the formation of virus-like particles. Journal of General Virology 79, 1047-1054).

For the purpose of achieving a biochemical characterization of the obtained material, Western blot experiments were carried out in which the different fractions were compared with specific sera against the VP1, pVP2, VP3 and VP4 proteins (Fernández-Arias et al. 1998, cited supra; Lombardo et al., 2000). Cell extracts infected with IBDV were used as a control. The obtained results are shown in FIG. 3d. As was expected, the bands corresponding to the VP1 and VP4 polypeptides were only detected in samples corresponding to cells infected with FBD/Poly-VP1. The patterns corresponding to pVP2/NVP3 in simples corresponding to cells infected with FBD/Poly-VP1 or coinfected with FB/VPX+ FB/his-VP3 were similar, two bands corresponding to pVP2 and VP3, respectively, being detected.

1.2 Obtaining IBDV CVLPs, pVP2-his-VP3, by Means of a Single rBV in Insect Cells

Furthermore, the construction of the plasmid pFBD/pVP2-his-VP3 was carried out. The first step of the construction was carried out by means of the cloning of the encoding region of the pVP2 protein into the pFBDual vector (Ivitrogen). The DNA fragment corresponding to pVP2 was obtained by means of PCR with the oligonucleotides identified as Oligo I (SEQ ID NO: 1) and Oligo II (SEQ ID NO: 2) using the plasmid pVOTE.2/Poly as a mold (Fernández-Arias, A., Risco, C., Martinez, S., Albar, J. P. & Rodriguez, J. F. (1998). Expression of ORF A1 of infectious bursal disease virus results in the formation of virus-like particles. Journal of General Virology 79, 1047-1054). The fragment was purified, subjected to digestion with the BglII and HindIII enzymes and cloned into the pFBDual vector (Invitrogen) previously digested with the BamHI and Hindi enzymes. The resulting plasmid was called pFBD/pVP2. Then, a DNA fragment containing the open reading frame corresponding to the VP3 protein was obtained by means of digestion of the plasmid pFB/his-VP3 (Kochan et al., 2003, cited supra) with the RsrII enzyme, treatment with Klenow, and subsequent restriction with KpnI. This DNA fragment was purified and cloned into the plasmid pFBD/pVP2 previously digested with the SmaI and KpnI enzymes. The resulting plasmid was called pFBD/pVP2-his-VP3 (SEQ ID NO: 3) and contains the encoding nucleotide sequence of the pVP2 proteins and of the his-pVP3 fusion protein containing a heterologous his 6 sequence (the latter is encoded by the complementary chain to the nucleotides 6734-7585 of SEQ ID NO: 3). The amino acid sequence of the pVP2 protein and of the his-VP3 fusion protein (pVP2-his-VP3) encoded by the nucleotide sequence contained in said plasmid pFBD/pVP2-his-VP3 is shown in SEQ ID NO: 4.

The plasmid pFBD/pVP2-his-VP3 allows obtaining an rBV, called FBD/pVP2-his-VP3, expressing both proteins simultaneously during its replication cycle [http://invitrogen.com/content/sfs/manuals/bevtest.pdf].

The results obtained with FBD/pVP2-his-VP3 in insect cells are identical to those obtained by means of the coinfection with rBVs FB/pVP2 and FD/his-VP3, IBDV CVLPs with the heterologous six histidine (6 his) polypeptide being obtained.

### EXAMPLE 2

### Obtaining IBDV CVLPs, pVP2-VP3-GFP, in Yeasts

For the purpose of studying the possibility of obtaining IBDV CVLPs in yeast cultures (Saccharomyces cerevisiae) the vector pESCURA/pVP2-VP3-GFP was generated with the heterologous GFP gene bound to the VP3 N-terminal end. The first step in the construction of the vector was carried out by means of the cloning of the encoding region of the pVP2 protein into the vector pESCURAinv. The plasmid pESCURAinv was generated by means of digestion of the vector pRS426 (Stratagene) with the PvuII enzyme and religation of the digestion mixture. The resulting vector, pESCURAinv, contains the multiple cloning region in reversed position with regard to that of parent vector pRS426. The DNA fragment corresponding to the pVP2 protein was obtained by means of PCR with the oligonucleotides called Oligo III (SEQ ID NO: 5) and Oligo IV (SEQ ID NO: 6) using the plasmid pVOTE.2/Poly as a mold (Fernández-Arias, A., Risco, C., Martinez, S., Albar, J. P. & Rodriguez, J. F. (1998). Expression of ORF A1 of infectious bursal disease virus results in the formation of virus-like particles. Journal of General Virology 79, 1047-1054). The fragment was purified subjected to digestion with the BglII and HindIII enzymes and cloned into the vector pESCURA.inv, previously digested with the BamHI and HindIII enzymes. The resulting plasmid was called pESCURA/pVP2.

The plasmid pFB/VP3-GFP was constructed in two stages. The first one consisted of the cloning of a DNA fragment, generated by means of PCR, containing the ORF of the VP3 protein lacking the termination codon. This PCR was carried out using the oligonucleotides called Oligo V (SEQ ID NO: 9) and Oligo VI (SEQ ID NO: 10) and using the plasmid pVOTE.2/Poly as a mold (Fernández-Arias, A., Risco, C., Martinez, S., Albar, J. P. & Rodriguez, J. F. (1998). Expression of ORF A1 of infectious bursal disease virus results in the formation of virus-like particles. Journal of General Virology 79, 1047-1054). The resulting DNA was digested with the EcoRI and BamHI enzymes and cloned into the vector pEGFP-N3 (Clontech), also digested with the same enzymes. The resulting plasmid was called pVP3-GFP. Then, the plasmid pEGFP-GFP was digested with the EcoRI and NotI enzymes and cloned into the vector pFastBac1 (Invitrogen). The resulting plasmid was called pFB/VP3-GFP.

Next, a DNA fragment that contained the open reading frame corresponding to the VP3 protein fused to the encoding region of the EGFP protein was obtained by means of digestion of the plasmid pFB/VP3-GFP with the EcoRI and NotI enzymes. This DNA fragment was purified and cloned into the plasmid pESCURA/pVP2 previously digested with the EcoRI and NotI enzymes. The resulting plasmid was called pESCURA/pVP2-VP3-GFP (SEQ ID NO: 7) and contains the ORFs of the pVP2 and VP3-GFP proteins under the transcriptional control of two independent promoters, GAL 1 and GAL 10, both inducible by galactose (the pVP2 protein is encoded by the chain of nucleotides complementary to the nucleotides 5862-7343 of SEQ ID NO: 7). The amino acid sequence of the pVP2 protein and of the VP3-GFP fusion protein (pVP2-VP3-GFP) encoded by the nucleotide sequence contained in said plasmid pESCURA/pVP2-VP3-GFP is shown in SEQ ID NO: 8.

pESCURA/pVP2-VP3-GFP was subsequently used to transform a culture of S. cerevisiae yeast haploid strain 499 according to a previously described protocol (Gietz, R.D. and R. A. Woods. (2002), Transformation of yeast by the Liac/SS carrier DNA/PEG method. Methods in Enzymology 350:87-96). The yeasts transformed with the plasmid were selected by means of growth on SC medium plates (CSM+YNB, 2% glucose and bacto agar) supplemented with the amino acids tryptophan, leucine and histidine and lacking uracyl (−Ura). After an incubation of 48 hours at 30° C., a colony was chosen which was used to carry out the following protein expression and CVLP formation analyses.

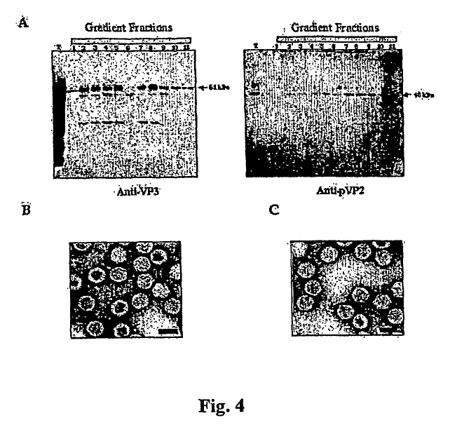

The pVP2 and VP3 protein expression and CVLP formation analyses were carried out following a protocol previously described for the characterization of IBDV VLPs in other expression systems (Fernández-Arias, A., Risco, C., Martinez, S., Albar, J. P. & Rodriguez, J. F. (1998). Expression of ORF A1 of infectious bursal disease virus results in the formation of virus-like particles. Journal of General Virology 79, 1047-1054; Lombardo, E., Maraver, A., Castón, J. R., Rivera, J., Fernández-Arias, A., Serrano, A., Carrascosa, J. L. & Rodriguez, J. F. (1999). VP1, the putative RNA-dependent RNA polymerase of infectious bursal disease virus, forms complexes with the capsid protein VP3, leading to efficient encapsidation into virus-like particles. Journal of Virology 73, 6973-698). The colony selected was cultured in liquid CSM (−Ura) +YNB medium supplemented with 2% raffinose. The culture was incubated at 30° C. for 24 hours. This culture was used to inoculate, at an optical density (O.D.) of 0.2, a flask of 200 ml of CSM (−Ura)+YNB medium supplemented with 2% inducer galactose. The culture was maintained at 30° C. for 18 hours (until an O.D. between 1.0 and 2.0). The yeasts were centrifuged at 3,000 radiant power measurement, 5 minutes at 4° C., were washed once with distilled water, and the pellet was resuspended in lysis buffer (TEN: Tris 10 mM, pH 8.0; NaCl 150 mM; EDTA 1 MM)+2×protease inhibitors (Compl Roche). A volume of glass beads having a size of about 425-600 microns (Sigma) were added for the lysis. This mixture was subjected to vigorous vortex stirring for 30 seconds 4 times, with 30-second intervals, and at 4° C. After this, the soluble fraction was recovered by centrifuging the lysis mixture at 13,000 rpm for 15 minutes at 4° C. This sample was subjected to fractioning on a sucrose gradient according to a previously described protocol (Lombardo, E., Maraver, A., Castón, J. R., Rivera, J., Fernández-Arias, A., Serrano, A., Carrascosa, J. L. & Rodriguez, J. F. (1999). VP1, the putative RNA-dependent RNA polymerase of infectious bursal disease virus, forms complexes with the capsid protein VP3, leading to efficient encapsidation into virus-like particles. Journal of Virology 73, 6973-6983). The samples obtained after fractioning as well as a sample of the starting material were analyzed by means of sodium dodecylsulfate polyacrylamide gel electrophoresis (SDS-PAGE) [Current Protocols in Molecular Biology] and immunodetection by Western blot (FIG. 4A) using anti-pVP2 and anti-VP3 sera [Current Protocols in Molecular Biology]. As is shown in FIG. 4A, the Western blot showed the presence of bands, with the predicted molecular mass corresponding to the pVP2 (48 kDa) and VP3-GFP (61 kDa) proteins, as well as other immunoreactive bands of a smaller size probably produced by proteolytic degradation both in the initial sample and in the different fractions of the gradient. These results reliably showed the correct expression of both polypeptides in the S. cerevisiae culture transformed with the plasmid pESCURA/pVP2-VP3. Then, the different fractions of the gradient were analyzed by means of TEM as has been previously described (Lombardo, B., Maraver, A., Castón, J. R., Rivera, J., Fernández-Arias, A., Serrano, A., Carrascosa, J. L. & Rodriguez, J. F. (1999). VP1, the putative RNA-dependent RNA polymerase of infectious bursal disease virus, forms complexes with the capsid protein VP3, leading to efficient encapsidation into virus-like particles. Journal of Virology 73, 6973-6983). As is shown in FIG. 4B, the TEM analysis of the fractions of the gradient showed the existence of IBDV CVLPs in the top fractions of the gradient. These CVLPs have a diameter of 65-70 nm and a polygonal contour that is indistinguishable from the IBDV CVLPs obtained in other expression systems (FIG. 4C).

### EXAMPLE 3

Obtaining and characterizing the immunogenicity of IBDV CVLPs As part of the development of new vaccination strategies, the possibility of using the strategy of producing chimeric IBDV VLPs (CVLPs) which contained heterologous amino acid sequences corresponding to other proteins or peptides involved in the induction of an immune response was analyzed. As a study model, the possibility of obtaining CVLPs which contained, as a heterologous polypeptide comprising a polypeptide of interest, the amino acid sequence corresponding to the CD8 epitope (E-CD8) of the malaria CS protein (Plasmodium yoelii), was approached. (Quantification of antigen specific CD8+T cells using an ELISPOT assay. J Immunol Methods 181: 45-54; Zavala, F., Rodrigues, M., Rodriguez, D., Rodriguez, J. R., Nussenzweig, R. S. and Esteban, M. (2001). A striking property of recombinant poxviruses: efficient inducers of in vivo expansion of primed CD8(+) T cells. Virology 280: 155-159). This epitope is responsible for the CD8-specific cellular immune response induction against this pathogen (Oliveira-Ferreira J, Miyahira Y, Layton G T, Savage N, Esteban M, Rodriguez D, Rodriguez J R, Nussenzweig R S, Zavala F, Myahira Y. (2000). Immunogenicity of Ty-VLP bearing a CD8(+) T cell epitope of the CS protein of P. yoelii: enhanced memory response by boosting with recombinant vaccinia virus. Vaccine 18: 1863-1869). This response can be quantified by means of the ELISPOT technique (Miyahira Y, Murata K, Rodriguez D, Rodriguez J R, Esteban M, Rodrigues M M, Zavala F. (1995) Quantification of antigen specific CD8+ T cells using an ELISPOT assay. J Immunol Methods 181: 45-54) in splenocyte cultures from BALB/c mice.

For this purpose, the construction of the plasmid pFB/his-CD8-VP3 (SEQ ID NO: 13) was carried out following the cloning strategy described later. This vector was constructed by means of insertion of a 36 bearing portion DNA fragment, generated by means of hybridization of the synthetic oligonucleotides identified as CD8 A (SEQ ID NO: 11) and CD8 B (SEQ ID NO: 12), containing the encoding sequence of the CD8 epitope (SYVPSAEQI, see residues 29 to 37 of the SEQ ID NO: 13 and 14) of the malaria CS protein in the ORF encoding the his-VP3 protein integrated in the pFB/his-VP3 vector. The cloning was carried out by means of ligation of the DNA fragment generated by means of hybridization of the synthetic oligonucleotides CD8 A and B (SEQ ID NO: 11 and SEQ ID NO: 12) to the plasmid pFB/his-VP3 digested with the EbeI restriction enzyme. This plasmid pFB/his-CD8-VP3 (SEQ ID NO: 13) contains and ORF encoding a fusion protein called his-CD8-VP3 containing the CD8 epitope inserted in the end corresponding to the N-terminal sequence of the his-VP3 protein ORF. The amino acid sequence of the his-CD8-VP3 fusion protein encoded by the nucleotide sequence contained in said plasmid pFB/his-CD8-VP3 is shown in SEQ ID NO: 14.

The plasmid pFB/his-CD8-VP3 was purified and used to generate the corresponding recombinant baculovirus (rBV), called FB/his-CD8-VP3, following the Bac-to-Bac technology according to the protocols described by the manufacturer (Invitrogen BV, Groningen, The Netherlands).

3.1 Producing CVLPs

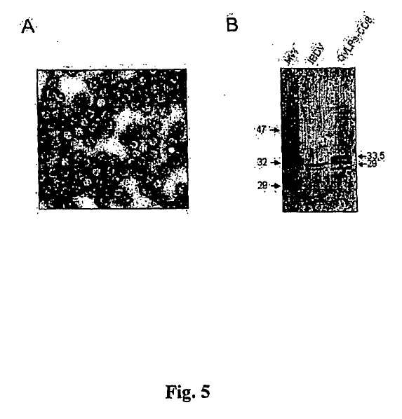

H5 cell cultures were, simultaneously infected with the recombinant baculoviruses FB/His-CD8-VP3 and FB/pVP2. The FB/pVP2 rBV (see Example 1.1) expresses the region corresponding to the pVP2 protein (Met1-Ala 512) of the IBDV polyprotein. The cells were harvested at 48 hours post-infection (pi), and the corresponding extracts were subjected to the IBDV VLPs purification protocol by means of fractioning on linear sucrose gradients (Lombardo, E., Maraver, A., Castón, J. R., Rivera, J., Fernández-Arias, A., Serrano, A., Carrascosa, J. L. & Rodriguez, J. P. (1999). VP1, the putative RNA-dependent RNA polymerase of infectious bursal disease virus, forms complexes with the capsid protein VP3, leading to efficient encapsidation into virus-like particles. Journal of Virology 73: 6973-6983). Each one of the obtained fractions was viewed by means of transmission electron microscopy (TEM) and analyzed by means of SDS-PAGE and immunoblot using VP3 specific antibody. As is observed in FIG. 5A, fraction 4 of the gradient contained abundant assemblies with an identical structure (polygonal perimeter and a diameter of 65-70 nm) as the IBDV VLPs obtained by means of expression of the viral polyprotein. The biochemical characterization, by means of SDS-PAGE and Western blot (FIG. 5B), showed that these CVLPs contain a protein, immunoreactive against the anti-VP3 serum, the molecular mass (33.5 kDa) of which is identical to the aforementioned one for the his-CD8-VP3 fusion protein (33.591 kDa). These results allow concluding that the coexpression of the pVP2 and his-CD8-VP3 genes in insect cells gives rise to the formation of chimeric VLPs (CVLPs) containing the his-CD8-VP3 fusion protein. These CVLPs are called CD8-CVLPs.

3.2 Immunogenicity Analysis of CD8-CVLPs

For the purpose of determining the immunogenic capacity of the CD8-CVLPs two identical assays were carried out using two batches of CD8-CVLPs independently produced and purified. Four groups (I, II, III and IV) of three female eight-week old BalbC rats were used. The groups were formed randomly. The immunization strategy was similar to the one used previously in the characterization of other immunogens. This strategy is based on the use of a priming dose with the antigen under study, followed by a second booster dose, which amplifies the primary response, with the recombinant vaccinia virus VVpJRCS, which expresses the malaria CS protein. The induced immune response was determined by means of the detection of the antigen specific CD8⁺ T cells according to their ability to produce IFN-γ, by means of an ELISPOT assay (Miyahira Y, Murata K, Rodriguez D, Rodriguez J R, Esteban M, Rodrigues M M, Zavala F. (1995). Quantification of antigen specific CD8+ T cells using an ELISPOT assay. J Immunol Methods 181: 45-54). In summary, 96-well plates with nitrocellulose (Millipore) bottoms were coated with 75 μl/well of a solution containing 6 μg/ml of the rat anti-murine IFN-y monoclonal antibody R4-6A2, Pharmingen, San Diego, Calif.) resuspended in PBS. The plates were incubated overnight at room temperature. The wells were subsequently washed three times with RPMI medium, and were finally incubated with RPMI medium supplemented with 10% fetal calf serum (FCS) for one hour at 37° C. 5% CO₂ atmosphere. On the other hand, the spleens of the immunized rats, maintained in RPMI medium supplemented with 10% FCS, were arranged on a sterile grid on a 60 movable member plate and were homogenized, the extract breaking up by means of its passing through needles of different gauges (21G->25G). The cells thus broken up were centrifuged for 5 minutes at 1,500 rpm at 4° C., and were washed twice with RPMI+10% FCS medium. In order to lysate the erythrocytes of the samples, sterile 0.1 M NH₄Cl (2 ml/spleen) was added and it was maintained at 4° C. for 3-5 minutes, RPMI+10% FCS was added and it was centrifuged. Then, they were twice and it was finally resuspended in 1-2 ml RPMI+10% FCS. The splenocyte viability count was carried out by means of trypan blue staining (4% in water, Sigma).

The professional antigen-presenting cells (APCs) used in this assay were P815. These cells were adjusted to a concentration of 106 cells/ml and were incubated with the synthetic peptide SYVPSAEQI (corresponding to the CD8 region of the malaria CS protein) 10⁻⁶ M. After treatment with the peptide, the cells were washed and treated with mitomycin C (30 μg/ml) (Sigma) for 15 minutes at 37° C. and in CO₂ atmosphere. After subsequent washings, the antigen-presenting cells, to which 30 U/ml of murine interleukin 2 (IL-2) were added, were added at a concentration of 10⁵ cells/well. 100 μl/well of 10⁶ splenocytes/ml and ¼ and 1/16 dilutions were also added. The plates were incubated for 18 ours at 37° C. in CO₂ atmosphere, they were washed 5 times with PBST and incubated with 2 μg/ml of the biotinylated rat anti-IFN-γ XMG1.2 monoclonal antibody (Pharmingen) diluted in PBST for 2 hours at room temperature. Then the plates were washed five times with PBST and a dilution of 1/800 avidin-peroxidase was added (0.5 mg/ml) (Sigma). After 1 hour of incubation at room temperature, it was washed 3 times with PBST and 2 times with PBS, finally adding the developer mixture with 1 μg/ml of the DAB substrate (Sigma), resuspended in Tris-HCl, pH 7.5, 50 mM, containing 0.015% H₂O₂. The reaction was stopped by washing the plate with abundant water, and once dried, the spots were counted with the aid of a Leica MZ122 APO stereomicroscope and the QWIN Imaging System software (Leica, Cambridge, United kingdom).

Immunizations were carried out according to the immunization program described in the following table:

| Group | 1^{st} Immunization. Day O | 2^{nd} Immunization. Day 14 |
|---|---|---|
| I | Not immunized | VVpJRPyCS |
| II | VVpJRPyCS | VVpJRPyCS |
| III | IBDV VLPs | VVpJRPyCS |
| IV | CD8-CVLPs | VVpJRPyCS |

Immunizations with VVpJRCS were carried out intraperitoneally using 10⁷ plaque forming units (pfu) per anima Immunizations with VLPs, both non-chimeric IBDV VLPs and CD8-CVLPs, were carried out intraperitoneally with a dose of 50 μg of antigen per animal. In all cases, the antigen preparations were diluted in phosphate-buffered saline (PBS).

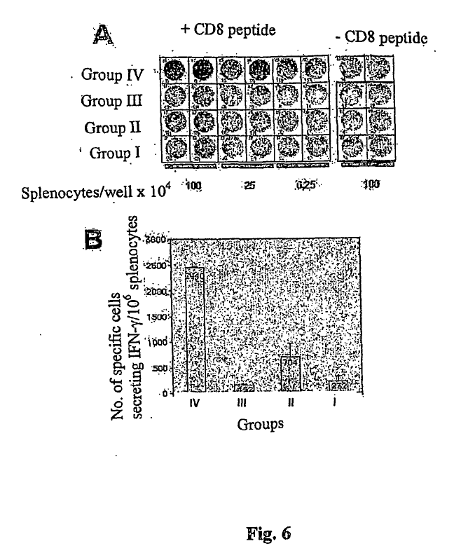

28 days after the first immunization, the animals were sacrificed and there spleens were used to carry out the ELISPOT assays. These assays were carried out following the protocol described above. Virtually identical results were obtained in both assays. FIG. 6 shows the results corresponding to the first assay. The obtained results demonstrate that when CD8-CVLPs are used as a priming dose followed by a booster dose with the VVpJRCS virus (group IV), a strong stimulation of the specific cellular immune response against the malaria CD8 epitope occurs. This stimulation is much greater (about 20 times greater) than that obtained after the immunization with one (group I) or two (group n)doses of VVpJRCS. The fact that a significant stimulation of the response against E-CD8 dose not occur in animals immunized with non-chimeric IBDV VLPs (group III), with regard to group I, which received a single dose of VVpJRCS, demonstrates that the response obtained in group IV is specifically induced by the E-CD8 present in the his-CD8-VP3 fusion protein forming an integral part of the CD8-CVLPs.

1. A chimeric empty capsid of the infectious bursal disease virus (IBDV), produced by assembly of a plurality of (i) IBDV pVP2 proteins and (ii) fusion proteins comprising a region A comprising an IBDV VP3 protein and region B comprising a heterologous polypeptide.

2. The chimeric empty capsid of claim 1, wherein said region B is bound to the amino-terminal region of IBDV VP3 or to the carboxy-terminal region of IBDV VP3.

3. The chimeric empty capsid of claim 1, wherein said polypeptide of interest is a polypeptide useful in vaccination, therapy or diagnosis.

4. The chimeric empty capsid of claim 1, wherein said region B comprises a single polypeptide of interest.

5. The chimeric empty capsid of claim 1, wherein said region B comprises two or more polypeptides of interest.

6. The chimeric empty capsid of claim 1, wherein said fusion protein comprises a region A bound to a single region B.

7. The chimeric empty capsid of claim 1, wherein said fusion protein comprises a region A bound to a first region B and a second region B, which first and second regions B are the same or different, wherein the first region B is bound to the amino-terminal region of the VP3 of region A and the second region B is bound to the carboxy-terminal region of the VP3 of in region A.

8. The chimeric empty capsid of claim 7, wherein said first and second regions B contain a plurality of heterologous polypeptides, which heterologous polypeptides are the same or different.

9. The chimeric empty capsid of claim 1, wherein said fusion protein further comprises a linker located between said regions A and B.

10. A nucleic acid, comprising a nucleotide sequence that encodes the fusion protein of claim 1.

11. The nucleic acid of claim 10, the open reading frame of one or more heterologous polypeptides comprising one or more wherein in the resulting fusion protein said heterologous polypeptide is bound to the amino-terminal region of IBDV VP3.

12. The nucleic acid of claim 11, further comprising (iii) a nucleotide sequence comprising an open reading frame corresponding to the IBDV pVP2 protein.

13. A gene construct comprising the nucleic acid of claim 10.

14. A gene construct comprising the nucleic acid of claim 12.

15. An expression system comprising the gene construct of claim 13 operatively bound to a transcription control element.

16. The expression system of claim 15, wherein said expression system is a plasmid, a bacmid, a yeast artificial chromosome (YAC), a bacteria artificial chromosome (BAC), a bacteriophage P1-based artificial chromosome (PAC), a cosmid, or a virus.

17. A host cell comprising the nucleic acid of claim 10.

18. (canceled)

19. The host cell of claim 17, said cell being selected from a mammal cell, an avian cell, an insect cell and a yeast.

20. A method for producing chimeric empty capsids of the infectious bursal disease virus (IBDV) comprising culturing a host cell comprising the nucleic acid of claim 10.

21. The method of claim 20, wherein said host cell is an insect cell and wherein the nucleic acid is a recombinant baculovirus.

22. The method of claim 20, wherein said host cell is a yeast and wherein the nucleic acid is a plasmid.

23. (canceled)

24. A medicament comprising the chimeric empty capsids of the infectious bursal disease virus (IBDV) of claim 1.

25. The medicament of claim 24, wherein said medicament is a vaccine.

26. The medicament of claim 24, wherein said medicament is a gene therapy vector.

27. A vaccine comprising a therapeutically effective amount of the chimeric empty capsids of the infectious bursal disease virus (IBDV) of claim 1 and one or more pharmaceutically acceptable adjuvants and/or vehicles.

28. The vaccine of claim 27, wherein the vaccine simultaneously protects at least one of an animal and a human against infection caused by two or more disease-causing infectious agents.

29. A gene therapy vector comprising the chimeric empty capsid of the infectious bursal disease virus (IBDV) of claim 1.

30. The expression system of claim 15, further comprising a second gene construct operatively bound to a transcription control element, said second gene construct comprising a nucleotide sequence comprising an open reading frame corresponding to the IBDV pVP2 protein.

31. An expression system comprising the gene construct of claim 14 operatively bound to a transcription control element.

32. The method of claim 20, further comprising recovering said chimeric empty IBDV capsids.


SEQUENCE LISTING (14 sequences)

SEQ ID NO: 1 - DNA, 35 bp, Artificial sequence
  Oligo I primer
gcgcagatctatgacaaacctgtcagatcaaaccc

SEQ ID NO: 2 - DNA, 34 bp, Artificial sequence
  Oligo II primer
gcgcaagcttaggcgagagtcagctgccttatgc

SEQ ID NO: 3 - DNA, 7595 bp, Artificial sequence
  Plasmid pFBD/pVP2-his-VP3
  promoter (157)..(285): Promoter ppolh
  CDS (291)..(1289): pVP2 ORF
  promoter (7443)..(7503): Promoter p10
gggtgatcaagtcttcgtcgagtgattgtaaataaaatgtaatttacagtatagtattttaattaatatacaaatgatttgataataattcttatttaactataatatattgtgttgggttgaattaaaggtccgtatactccggaatattaatagatcatggagataattaaaatgataaccatctcgcaaataaataagtattttactgttttcgtaacagttttgtaataaaaaaacctataaatattccggattattcataccgtcccaccatcgggcgcggatctatgacaaacctgtcagatcaaacccagcagattgttccgttcatacggagccttctgatgccaacaaccggaccggcgtccattccggacgacaccctggagaagcacactctcaggtcagagacctcgacctacaatttgactgtgggggacacagggtcagggctaattgtctttttccctggattccctggctcaattgtgggtgctcactacacactgcagggcaatgggaactacaagttcgatcagatgctcctgactgcccagaacctaccggccagttacaactactgcaggctagtgagtcggagtctcacagtgaggtcaagcacacttcctggtggcgtttatgcactaaacggcaccataaacgccgtgaccttccaaggaagcctgagtgaactgacagatgttagctacaatgggttgatgtctgcaacagccaacatcaacgacaaaattgggaacgtcctagtaggggaaggggtcaccgtcctcagcttacccacatcatatgatcttgggtatgtgaggcttggtgaccccattcccgcaatagggcttgacccaaaaatggtagccacatgtgacagcagtgacaggcccagagtctacaccataactgcagccgatgattaccaattctcatcacagtaccaaccaggtggggtaacaatcacactgttctcagccaacattgatgccatcacaagcctcagcgttgggggagagctcgtgtttcgaacaagcgtccacggccttgtactgggcgccaccatctacctcataggctttgatgggacaacggtaatcaccagggctgtggccgcaaacaatgggctgacgaccggcaccgacaaccttatgccattcaatcttgtgattccaacaaacgagataacccagccaatcacatccatcaaactggagatagtgacctccaaaagtggtggtcaggcaggggatcagatgtcatggtcggcaagagggagcctagcagtgacgatccatggtggcaactatccaggggccctccgtcccgtcacgctagtggcctacgaaagagtggcaacaggatccgtcgttacggtcgctggggtgagcaacttcgagctgatcccaaatcctgaactagcaaagaacctggttacagaatacggccgatttgacccaggagccatgaactacacaaaattgatactgagtgagagggaccgtcttggcatcaagaccgtctggccaacaagggagtacactgactttcgtgaatacttcatggaggtggccgacctcaactctcccctgaagattgcaggagcattcggcttcaaagacataatccgggccataaggaggatagctgtgccggtggtctccacattgttcccacctgccgctcccctagcccatgcaattggggaaggtgtagactacctgctgggcgatgaggcccaggccgcttcaggaactgctcgagccgcgtcaggaaaagcaagagctgcctcaggccgcataaggcagctgactctcgcctaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgcctcgagactgcaggctctagattcgaaagcggccgcgactagtgagctcgtcgacgtaggcctttgaattccggatcctcactcaaggtcctcatcagagacggtcctgatccagcggcccagccgaccagggggtctctgtgttggagcattgggttttggcttgggctttggtagagcccgcctgggattgcgatgcttcatctccatcgcagtcaagagcagatctttcatctgttcttggtttgggccacgtccatggttgatttcatagactttggcaacttcgtctatgaaagcttggggtggctctgcctgtcctggagccccgtagatcgacgtagctgcccttaggatttgttcttctgatgccaaccggctcttctctgcatgcacgtagtctagatagtcctcgtttgggtccggtatttctcgtttgttctgccagtactttacctggcctgggcttggccctcggtgcccattgagtgctacccattctggtgttgcaaagtagatgcccatggtctccatcttctttgagatccgtgtgtctttttccctctgtgcttcctctggtgtggggccccgagcctccactccgtagcctgctgtcccgtacttggccctttgcgacttgctgcctgcttgtggtgcgtttgcaagaaaatttcgcatccgatgggcgttcgggtcgctgagtgcgaagttggccatgtcagtcacaatcccattctcttccagccacatgaacacactgagtgcagattggaatagtgggtccacgttggctgctgcttccattgctctgacggcactctcgagttcgggggtctctttgaactctgatgcagccatggcgccctgaaaatacaggttttcggtcgttgggatatcgtaatcgtgatggtgatggtgatggtagtacgacatggtttcggac

SEQ ID NO: 4 - protein, 333 aa, Artificial sequence
  pVP2-his-VP3 protein
MTNLSDQTQQIVPFIRSLLMPTTGPASIPDDTLEKHTLRSETSTYNLTVGDTGSGLIVFFPGFPGSIVGAHYTLQGNGNYKFDQMLLTAQNLPASYNYCRLVSRSLTVRSSTLPGGVYALNGTINAVTFQGSLSELTDVSYNGLMSATANINDKIGNVLVGEGVTVLSLPTSYDLGYVRLGDPIPAIGLDPKMVATCDSSDRPRVYTITAADDYQFSSQYQPGGVTITLFSANIDAITSLSVGGELVFRTSVHGLVLGATIYLIGFDGTTVITRAVAANNGLTTGTDNLMPFNLVIPTNEITQPITSIKLEIVTSKSGGQAGDQMSWSARGSL

SEQ ID NO: 5 - DNA, 35 bp, Artificial sequence; identical to SEQ ID NO: 1
  Oligo III primer

SEQ ID NO: 6 - DNA, 34 bp, Artificial sequence; identical to SEQ ID NO: 2
  Oligo IV primer

SEQ ID NO: 7 - DNA, 9600 bp, Artificial sequence
  Plasmid pESCURA/pVP2-VP3-GFP
  promoter (5649)..(5859): Promoter 1 (pVP2)
  promoter (7402)..(8080): Promoter 2 (VP3-GFP)
  CDS (8086)..(9597): VP3-GFP ORF
ggccgcactagtatcgatggattacaaggatgacgacgataagatctgagctcttaattaacaattcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctccaattcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggatcttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatcttagctagccgcggtaccaagcttaggcgagagtcagctgccttatgcggcctgaggcagctcttgcttttcctgacgcggctcgagcagttcctgaagcggcctgggcctcatcgcccagcaggtagtctacaccttccccaattgcatgggctaggggagcggcaggtgggaacaatgtggagaccaccggcacagctatcctccttatggcccggattatgtctttgaagccgaatgctcctgcaatcttcaggggagagttgaggtcggccacctccatgaagtattcacgaaagtcagtgtactcccttgttggccagacggtcttgatgccaagacggtccctctcactcagtatcaattttgtgtagttcatggctcctgggtcaaatcggccgtattctgtaaccaggttctttgctagttcaggatttgggatcagctcgaagttgctcaccccagcgaccgtaacgacggatcctgttgccactctttcgtaggccactagcgtgacgggacggagggcccctggatagttgccaccatggatcgtcactgctaggctccctcttgccgaccatgacatctgatcccctgcctgaccaccacttttggaggtcactatctccagtttgatggatgtgattggctgggttatctcgtttgttggaatcacaagattgaatggcataaggttgtcggtgccggtcgtcagcccattgtttgcggccacagccctggtgattaccgttgtcccatcaaagcctatgaggtagatggtggcgcccagtacaaggccgtggacgcttgttcgaaacacgagctctcccccaacgctgaggcttgtgatggcatcaatgttggctgagaacagtgtgattgttaccccacctggttggtactgtgatgagaattggtaatcatcggctgcagttatggtgtagactctgggcctgtcactgctgtcacatgtggctaccatttttgggtcaagccctattgcgggaatggggtcaccaagcctcacatacccaagatcatatgatgtgggtaagctgaggacggtgaccccttcccctactaggacgttcccaattttgtcgttgatgttggctgttgcagacatcaacccattgtagctaacatctgtcagttcactcaggcttccttggaaggtcacggcgtttatggtgccgtttagtgcataaacgccaccaggaagtgtgcttgacctcactgtgagactccgactcactagcctgcagtagttgtaactggccggtaggttctgggcagtcaggagcatctgatcgaacttgtagttcccattgccctgcagtgtgtagtgagcacccacaattgagccagggaatccagggaaaaagacaattagccctgaccctgtgtcccccacagtcaaattgtaggtcgaggtctctgacctgagagtgtgcttctccagggtgtcgtccggaatggacgccggtccggttgttggcatcagaaggctccgtatgaacggaacaatctgctgggtttgatctgacaggtttgtcatagatccggggttttttctccttgacgttaaagtatagaggtatattaacaattttttgttgatacttttattacatttgaataagaagtaatacaaaccgaaaatgttgaaagtattagttaaagtggttatgcagtttttgcatttatatatctgttaatagatcaaaaatcatcgcttcgctgattaattaccccagaaataaggctaaaaaactaatcgcattatcatcctatggttgttaatttgattcgttcatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccttcggagggctgtcacccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggatatgtatatggtggtaatgccatgtaatatgattattaaacttctttgcgtccatccaaaaaaaaagtaagaatttttgaaaattcgaattcgatggctgcatcagagttcaaagagacccccgaactcgagagtgccgtcagagcaatggaagcagcagccaacgtggacccactattccaatctgcactcagtgtgttcatgtggctggaagagaatgggattgtgactgacatggccaacttcgcactcagcgacccgaacgcccatcggatgcgaaattttcttgcaaacgcaccacaagcaggcagcaagtcgcaaagggccaagtacgggacagcaggctacggagtggaggctcggggccccacaccagaggaagcacagagggaaaaagacacacggatctcaaagaagatggagaccatgggcatctactttgcaacaccagaatgggtagcactcaatgggcaccgagggccaagcccaggccaggtaaagtactggcagaacaaacgagaaataccggacccaaacgaggactatctagactacgtgcatgcagagaagagccggttggcatcagaagaacaaatcctaagggcagctacgtcgatctacggggctccaggacaggcagagccaccccaagctttcatagacgaagttgccaaagtctatgaaatcaaccatggacgtggcccaaaccaagaacagatgaaagatctgctcttgactgcgatggagatgaagcatcgcaatcccaggcgggctctaccaaagcccaagccaaaacccaatgctccaacacagagaccccctggtcggctgggccgctggatcaggaccgtctctgatgaggaccttgagggatccatcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagc

SEQ ID NO: 8 - protein, 503 aa, Artificial sequence
  pVP2-VP3-GFP protein
MAASEFKETPELESAVRAMEAAANVDPLFQSALSVFMWLEENGIVTDMANFALSDPNAHRMRNFLANAPQAGSKSQRAKYGTAGYGVEARGPTPEEAQREKDTRISKKMETMGIYFATPEWVALNGHRGPSPGQVKYWQNKREIPDPNEDYLDYVHAEKSRLASEEQILRAATSIYGAPGQAEPPQAFIDEVAKVYEINHGRGPNQEQMKDLLLTAMEMKHRNPRRALPKPKPKPNAPTQRPPGRLGRWIRTVSDEDLEGSIATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 9 - DNA, 33 bp, Artificial sequence
  Oligo V primer
gcgcgaattcgatggcatcagagttcaaagaga

SEQ ID NO: 10 - DNA, 32 bp, Artificial sequence
  Oligo VI primer
cgcggatccctcaaggtcctcatcagagacgg

SEQ ID NO: 11 - DNA, 36 bp, Artificial sequence
  Oligo CD8 A primer
aacgaggacagttatgtcccaagcgcagaacaaata

SEQ ID NO: 12 - DNA, 36 bp, Artificial sequence
  Oligo CD8 B primer
tatttgttctgcgcttgggacataactgtcctcgtt

SEQ ID NO: 13 - DNA, 5676 bp, Artificial sequence
  Plasmid pFB/his-CD8-VP3
  promoter (1)..(129): Polyhedrin promoter
  CDS (147)..(1043): His-CD8-VP3 ORF
  CDS (222)..(257): His-CD8 ORF
atcatggagataattaaaatgataaccatctcgcaaataaataagtattttactgttttcgtaacagttttgtaataaaaaaacctataaatattccggattattcataccgtcccaccatcgggcgcggatctcggtccgaaaccatgtcgtactaccatcaccatcaccatcacgattacgatatcccaacgaccgaaaacctgtattttcagggcgcgaacgaggacagttatgtcccaagcgcagaacaaatagccgccatggctgcatcagagttcaaagagacccccgaactcgagagtgccgtcagagcaatggaagcagcagccaacgtggacccactattccaatctgcactcagtgtgttcatgtggctggaagagaatgggattgtgactgacatggccaacttcgcactcagcgacccgaacgcccatcggatgcgaaattttcttgcaaacgcaccacaagcaggcagcaagtcgcaaagggccaagtacgggacagcaggctacggagtggaggctcggggccccacaccagaggaagcacagagggaaaaagacacacggatctcaaagaagatggagaccatgggcatctactttgcaacaccagaatgggtagcactcaatgggcaccgagggccaagcccaggccaggtaaagtactggcagaacaaacgagaaataccggacccaaacgaggactatctagactacgtgcatgcagagaagagccggttggcatcagaagaacaaatcctaagggcagctacgtcgatctacggggctccaggacaggcagagccaccccaagctttcatagacgaagttgccaaagtctatgaaatcaaccatggacgtggcccaaaccaagaacagatgaaagatctgctcttgactgcgatggagatgaagcatcgcaatcccaggcgggctctaccaaagcccaagccaaaacccaatgctccaacacagagaccccctggtcggctgggccgctggatcaggaccgtctctgatgaggaccttgagtgaggatccggaattcaaaggcctacgtcgacgagctcactagtcgcggccgctttcgaatctagagcctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactagatctgcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatag

SEQ ID NO: 14 - protein, 298 aa, Artificial sequence
  his-CD8-VP3 protein
MSYYHHHHHHDYDIPTTENLYFQGANEDSYVPSAEQIAAMAASEFKETPELESAVRAMEAAANVDPLFQSALSVFMWLEENGIVTDMANFALSDPNAHRMRNFLANAPQAGSKSQRAKYGTAGYGVEARGPTPEEAQREKDTRISKKMETMGIYFATPEWVALNGHRGPSPGQVKYWQNKREIPDPNEDYLDYVHAEKSRLASEEQILRAATSIYGAPGQAEPPQAFIDEVAKVYEINHGRGPNQEQMKDLLLTAMEMKHRNPRRALPKPKPKPNAPTQRPPGRLGRWIRTVSDEDLE